(19) United States
(12) Patent Application Publication
(10) Pub. No.: US 2018/0333447 A1
(43) Pub. Date: Nov. 22, 2018

(54) BENEFICIAL EFFECTS OF BIDENS PILOSA ON GUT MICROFLORA AND ANIMAL HEALTH

(71) Applicant: Academia Sinica, Taipei City (TW)

(72) Inventors: Wen-Chin YANG, Taichung County (TW); Lee-Tian CHANG, Taichung (TW)

(21) Appl. No.: 15/777,654

(22) PCT Filed: Nov. 21, 2016
(86) PCT No.: PCT/US2016/063189

§ 371 Date: May 19, 2018

Related U.S. Application Data
(60) Provisional application No. 62/260,499, filed on Nov. 28, 2015.

(51) Int. Cl. A61K 36/28; A61K 31/7028; A61K 9/14; A61P 1/00; A23K 10/30; A23K 50/75

(52) U.S. Cl. CPC A61K 36/28; A61K 31/7028; A23K 50/75; A61P 1/00; A23K 10/30; A61K 9/14

(57) ABSTRACT

Beneficial effects of Bidens pilosa on gut microflora and animal health. A composition comprising a therapeutically effective amount of Bidens pilosa extract or an active compound isolated from the Bidens pilosa extract for use in promoting beneficial gut microbiota and/or inhibiting pathogenic gut microbiota in an animal in need thereof is disclosed. In one embodiment, the composition is for use in promoting growth performance or increasing body weight in an animal in need thereof. In another embodiment, the composition is for use in an animal that is not afflicted with coccidiosis.

### FIELD OF THE INVENTION

The present invention relates generally to compositions for use in promoting and/or improving gut health, and more specifically to compositions for use in modulating gut microbiota with a prebiotic.

### BACKGROUND OF THE INVENTION

It was estimated that 50 billion chickens are raised in the world, reaching a global market value of 60 billion American dollars. Chicken meat accounts for 30% of protein food consumed by humans. Gut health determines growth performance and health in chickens because the gastrointestinal tract, the main digestive and absorption organ, can take in nutrients for growth and development, eliminate unwanted waste, and confer mucosal immunity against parasites. A diverse microbiota is found throughout the digestive tract and is more profound in the cecum. Gut microbiota affects nutrition, detoxification, growth performance, and protection against pathogens in chickens. Therefore, gut microbiota are important for gut health and diseases in chickens.

Plants have been an extraordinary source of medicines for humans and animals since antiquity. Edible plants and their compounds have become an alternative approach to treat intestinal parasites. The herbal approach can reduce or replace the abuse and misuse of antibiotics in chickens and help organic chicken production.

### SUMMARY OF THE INVENTION

In one aspect, the invention relates to a composition comprising a therapeutically effective amount of Bidens pilosa extract or an active compound isolated from the Bidens pilosa extract for use in promoting beneficial gut microbiota and/or inhibiting pathogenic gut microbiota in an animal in need thereof.

Alternatively, the invention relates to use of a composition comprising a therapeutically effective amount of Bidens pilosa extract, or an active compound isolated from the Bidens pilosa extract in the manufacture of a medicament for promoting beneficial gut microbiota and/or inhibiting pathogenic gut microbiota in an animal in need thereof.

Additionally, the invention relates to a method for promoting beneficial gut microbiota and/or inhibiting pathogenic gut microbiota in an animal in need thereof, comprising administering to the animal in need thereof a therapeutically effective amount of Bidens pilosa extract or an active compound isolated from the Bidens pilosa extract.

In another embodiment, the animal is subjected to, or the method may further comprises at least one of the following steps:
(i) performing examination of the animal gut health;
(ii) performing examination of the animal gut structure with X ray, CT scan, gut endoscopy;
(iii) performing examination of gut pathology of the animal; and
(iv) performing examination of crypt, villi, gut integrity, leukocyte infiltration, and/or inflammation.

In one embodiment, the animal is not afflicted with coccidiosis or not infected by E. tenella.

In another embodiment, the animal is in need of promoting gut health.

In another embodiment, the animal is in need of gaining body weight.

In another embodiment, the animal is selected from the group consisting of humans, non-human mammals, fish, birds, and reptiles.

In another embodiment, the beneficial gut micribiota comprise at least one bacteria genus selected from the group consisting of Bacteroides, Megamonas, Rikenella, and Ruminococcus2, Alistipes, Bilophila and Lactobacillus.

In another embodiment, the pathogenic gut microbiota are at least one bacteria genus selected from the group consisting, of Actinobacter, Clostridium IV, Anaerostipes, Anaeroplasma, Enterococcus, Campylobacteria, Flavonifractor, Escherichia/Shigella, Oscillibacter, PseodoFlavonifractor, Odoribacter, Phascolarctobacterium, Anaerotruncus, Butyricicoccus, and Clostridium XIVb.

In another embodiment, the composition is in a dosage form selected from the group consisting of oral, capsule, suppository and parenteral.

In another embodiment, the active compound isolated from the Bidens pilosa extract is a polyacetylenic compound of formula (I):

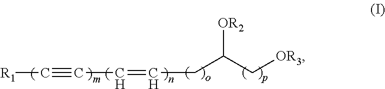

wherein
   R₁ is H or CH3;
   R₂ is monosaccharide;
   R₃ is H or COCH₂COOH;
   m=3 or 4;
   n=0 or 1;
   o=1 or 2; and
   p=1 or 2.

In another embodiment, the active compound is selected from the group consisting of

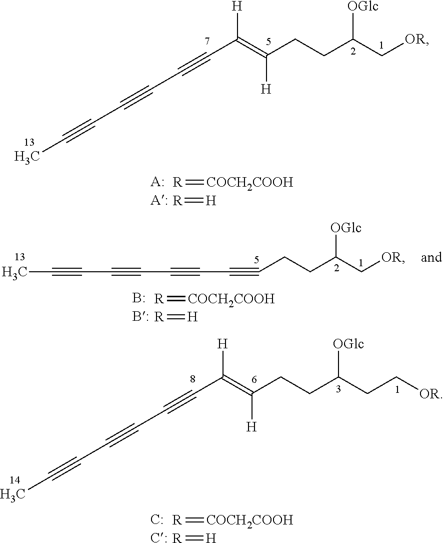

In another embodiment, the effective amount of the active compound isolated from the Bidens pilosa extract is at a dose of no less than 1 μg/kg body weight of the animal in need thereof.

In another embodiment, the composition comprises the animal feed and 0.0005%˜15% (w/w) of Bidens pilosa extract.

In another embodiment, the Bidens pilosa extract is in a form of powder.

In another embodiment, the composition further comprises an animal feed.

These and other aspects will become apparent from the following description of the preferred embodiment taken in conjunction with the following drawings, although variations and modifications therein may be affected without departing from the spirit and scope of the novel concepts of the disclosure.

The accompanying drawings illustrate one or more embodiments of the invention and, together with the written description, serve to explain the principles of the invention. Wherever possible, the same reference numbers are used throughout the drawings to refer to the same or like elements of an embodiment.

### BRIEF DESCRIPTION OF THE DRAWINGS

FIG. 1A shows experimental schemes.

FIG. 1B shows representative images of the gut of 21-day-old chickens in each group.

FIG. 1C shows hematoxylin and esosin (HE) staining of the coca of the chickens in FIG. 1B.

FIG. 1D shows hematoxylin and esosin (HE) staining of the jejuna of the chickens in FIG. 1B.

FIG. 2 shows rarefaction curves of bacterial OTUs in experimental samples from the guts of chickens fed with or without B. pilosa, infected with or without E. tenella. Rarefaction curves of bacterial 16S rRNA sequences from the guts of 18-day-old (4D) and 21-day-old (7D) chickens of FIG. 1B fed with or without B. pilosa, infected with or without E. tenella were analyzed.

FIG. 3 shows the result of principal component analysis of the bacterial community compositions at the genus level in the guts of chickens. Principal component analysis was conducted to compare the bacteria genera based on 16S rRNA sequences in the 8 samples of FIG. 2.

FIG. 4 shows the proportion of the bacterial community compositions at the genus level in the guts of chickens. The proportion of the bacterial genera in the guts of chickens in FIG. 2 was determined. Each bacterial genus is indicated by a number code.

FIG. 5 shows the result of clustering analysis of the compositions of the bacterial genera in the guts of chickens. Clustering analysis of the bacterial genera in chicken guts of FIG. 2 was performed to correlate the proportion of the bacterial community with their genera.

FIGS. 6A-B show chances in the proportion of the probiotic bacterial genera in the guts of chickens with or without B. pilosa. The bacterial genera that showed an increase in the proportion in the guts of the non-infected chickens by B. pilosa (from FIG. 2) were re-plotted into histograms (FIGS. 6A-B). Only the data obtained from CTR_4D, BPP_4D, CTR_7D and BPP_D are shown. Two patterns of change in the proportions of the bacterial genera are presented. The genera in FIG. 6A were increased in 18-day-old chickens whereas those in FIG. 6B were increased in 21-day-old chickens.

FIG. 7 shows changes in the proportion of the zoonotic bacterial genera in the guts of chickens, fed with or without B. pilosa, following E. tenella infection. The bacterial genera, which decreased in the proportion in the guts of the E. tenella-infected chickens by B. pilosa (from FIG. 2), were re-plotted into histograms.

### DETAILED DESCRIPTION OF THE INVENTION

### Definitions

The terms used in this specification generally have their ordinary meanings in the art, within the context of the invention, and in the specific context where each term is used. Certain terms that are used to describe the invention are discussed below, or elsewhere in the specification, to provide additional guidance to the practitioner regarding the description of the invention. For convenience, certain terms may be highlighted, for example using italics and/or quotation marks. The use of highlighting has no influence on the scope and meaning of a term; the scope and meaning of a term is the same, in the same context, whether or not it is highlighted. It will be appreciated that same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of other synonyms. The use of examples anywhere in this specification including examples of any terms discussed herein is illustrative only, and in no way limits the scope and meaning of the invention or of any exemplified term. Likewise, the invention is not limited to various embodiments given in this specification.

Unless otherwise defined, all technical and scientific terms used herein have the same meaning as commonly understood by one of ordinary skill in the art to which this invention pertains. In the case of conflict, the present document, including definitions will control.

As used herein, “around”, “about” or “approximately” shall generally mean within 20 percent, preferably within 10 percent, and more preferably within 5 percent of a given value or range. Numerical quantities given herein are approximate, meaning that the term “around”, “about” or “approximately” can be inferred if not expressly stated.

The term “alkyl” refers to a saturated, linear or branched, non-aromatic hydrocarbon moiety, such as CH₃, —CH₂—, or branched (CH₃)₂CH₂—. The term “alkenyl” refers to a linear or branched, non-aromatic hydrocarbon moiety having at least one double bond, such as CH₂═CH—, or —CH═CH—. The term “alkynyl” refers to a linear or branched, non-aromatic hydrocarbon moiety having a least one triple bond, such as CH≡C— or —C≡C—. The term “cycloalkyl” refers to a saturated non-aromatic cyclic hydrocarbon moiety, such as cyclohexyl. The term “cycloalkenyl” refers to a non-aromatic, cyclic hydrocarbon moiety that contains at least one double bond in the ring, such as cyclohexenyl, The term “heterocycloalkyl” refers to a saturated non-aromatic, cyclic moiety having at least one ring heteroatom (e.g., O, N, S), such as 4-tetrahydropyranyl. The term “heterocycloalkenyl” refers to a non-aromatic, cyclic moiety having at least one ring heteroatom and at least one double bond in the ring, such as pyranyl. The term “aryl” refers to a hydrocarbon moiety having at least one aromatic ring. Examples of aryl moieties include phenyl, phenylene, biphenyl, naphthyl, naphthylene, pyrenyl, anthryl, and phenanthryl. The term “heteroaryl” refers to a moiety having at least one aromatic ring which contains at least one heteroatom. Examples of heteroaryl moieties include furyl, furylene, fluorenyl, pyrrolyl, thienyl, oxazolyl, thizolyl, pyridyl, pyrimidinyl, quinazolinyl, isoquinolyl, and indolyl.

Alkyl, alkenyl, alkynyl, cycloalkyl, cycloalkenyl, heterocycloalkyl, heterocycloalkenyl, aryl, and heteroaryl mentioned herein include both substituted and unsubstituted moieties. Examples of substituents on cycloalkyl, cycloalkenyl, heterocycloalkyl, heterocycloalkenyl, aryl, and heteroaryl include, but are not limited to, C₁-C₁₀ alkyl, C₂-C₁₀ alkenyl, C₃-C₂₀ alkynyl, C₃-C₂₀ cycloalkyl, C₃-C₂₀ cycloalkenyl, alkoxy, aryl, aryloxy, heteroaryl, heteroaryloxy, amino, C₁-C₁₀ alkylamino, C₁-C₂₀ dialkylamino, arylamino, diarylamino, heteroarylamino, diheteroarylamino, C₁-C₁₀ alkylsulfony, arylsulfonyl, heteroarylsulfonyl, C₁-C₁₀ alkylsulfonamide, arylsulfonamide, heteroarylsulfonamide C₁-C₁₀ alkylimino, arylimino, C₁-C₁₀ alrylsulfonimino, hydroxyl halo, thio, C₁-C₁₀ alkylthio, arylthio, aminothioacyl, amidino, guanidine, ureido, cyano, nitro, nitroso, azido, acyl, thioacyl, acyloxy, carboxyl, amido, carbamoyl, and carboxyl, and carboxylic ester. Examples of substituents on alkyl, alkenyl, and alkynyl include all of the above-recited substituents except C₁-C₁₀ alkyl, C₂-C₁₀ alkenyl, and C₂-C₁₀ alkynyl. Cycloalkyl, cycloalkenyl, heterocycloalkyl, heterocycloalkenyl, aryl, and heteroaryl can also be fused with each other.

An animal feed refers to food given to domestic livestock, and pet (companion animal) food.

The term “pure compound” used herein refers to a compound that has a purity of at least 80% (e.g., 95% or 99%).

The term “treating” or “treatment” refers to administration of an effective amount of Bidens pilosa or its phytochemicals polyacetylenic compounds such as cytopiloyne) to a subject, who has coccidosis, or a symptom or predisposition toward such a disease, with the purpose to cure, alleviate, relieve, remedy, ameliorate, or prevent coccidosis, the symptoms of it, or the predispositions towards it.

As used herein, “effective amount” or “sufficient amount” of Bidens pilosa or a compound refers to an amount that may be therapeutically effective to enhance growth, and/or inhibit, prevent, or treat a symptom of a particular disease, disorder, condition, or side effect described herein. For example, “an effective amount” may refer to the amount that is required to confer a therapeutic or a desired effect on the treated subject. Effective doses will vary, as recognized by those skilled in the art, depending on route of administration, excipient usage, and the possibility of co-usage with other therapeutic treatment.

The “Guidance for Industry and Reviewers Estimating the Safe Starting Dose in Clinical Trials for Therapeutics in Adult Healthy Volunteers” published by the U.S. Department of Health and Human Services Food and Drug Administration discloses “a human equivalent dose” may be obtained by calculations from the following formula:

HED=animal dose in mg/kg×(animal weight in kg/human weight in kg)^{0.33}.

B. pilosa powder was prepared first. Then animal diets were formulated by mixing with different percentages of B. pilosa powder.

Bidens pilosa preparation. Such a preparation can be obtained by stirring pulverized Bidens pilosa plants in water at an elevated temperature (e.g., at 50° C. or 100° C.) to form a suspension, and collecting a supernatant of the suspension. The supernatant can be further extracted with an alcohol (e.g., n-butanol) to provide an enriched preparation. The Bidens pilasa preparation contains one or more of the polyacetylenic compounds of the just-mentioned formula (I). For example, it contains cytopiloyne:

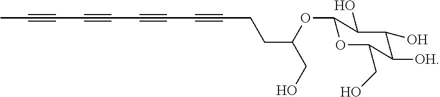 [SMILES: CC#CC#CC#CC#CCCC(CO)OC1OC(CO)C(O)C(O)C1O]

The polyacetylenic compounds described above include the compounds themselves, as well as their salts, prodrugs, and solvates, if applicable. Such salts, for example, can be formed by interaction between a negatively charged substituent carboxylate) on a polyacetylenic compound and a cation. Suitable cations include, but are not limited to, sodium ion, potassium ion, magnesium ion, calcium ion, and an ammonium cation (e.g., tetramethylammonium ion). Likewise, a positively charged substituent (e.g., amino) on a polyacetylenic compound can form a salt with a negatively charged counter ion. Suitable counter ions include, but are not limited to, chloride, bromide, iodide, sulfate, nitrate, phosphate, or acetate. Examples of prodrugs include esters and other pharmaceutically acceptable derivatives, which, upon administration to a subject, are capable of providing above compounds described above. A solvate refers to a complex formed between a polyacetylenic compound and a pharmaceutically acceptable solvent. Examples of pharmaceutically acceptable solvents include water, ethanol, isopropanol, n-butanol, ethyl acetate, and acetic acid.

The polyacetylenic compounds may contain one or more asymmetric centers or a non-aromatic double bond. Thus, they can occur as racemates and racemic mixtures, single enantiomers, individual diastereomers, diastereomeric mixtures, and cis- or trans-isomeric forms. All such isomeric forms are contemplated.

### Polyacetylenic Compounds

Polyacetylenic compounds (e.g. cytopiloyne) can be isolated from Bidens pilosa. Whole Bidens pilosa plants are first pulverized and then stirred in heated water. After removal of insoluble materials (e.g., by filtration, decantation, or centrifugation), the resultant supernatant is subjected to liquid chromatography (e.g., high-pressure liquid chromatography) or other suitable methods to afford pure polyacetylenic compounds. The pure compounds thus obtained can be further derivatized to provide a number of other polyacetylenic compounds of this invention (U.S. Pat. No 7,763,285, and Kusano et al (JP 2004083463), all of which are incorporated herein by reference in their entireties).

The polyacetylenic compounds described above can also be prepared by conventional methods. Below are three reaction schemes illustrating synthetic routes to a polyacetylenic compound of this invention.

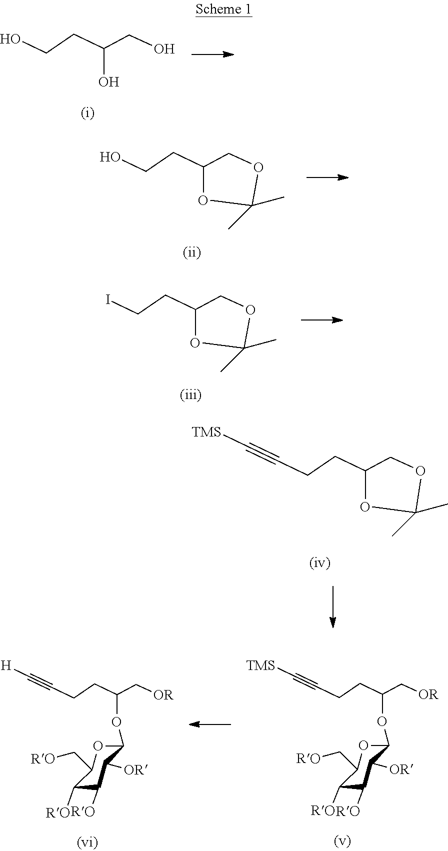

Butane-1,2,4-triol (i) is reacted with acetone to form a protected 1,2,4-triol compound (ii), which can be readily transformed to an iodo derivative (iii). Compound (iii) is then reacted with ethynyltrimethylsilane, under a basic condition (e.g., n-Buli), to give (4-(2,2-dimethyl-1,3-dioxolan-4-yl)but-1-ynyl)trimethylsilane (iv), Compound (iv) is subsequently treated with an acid (e.g., acetic acid), followed by a coupling reaction with 2-bromoglucopyranose to afford an adduct (v). Compound (v) can be further treated with potassium fluoride to afford 2-phenyl-4H-chromen-4-one (vi).

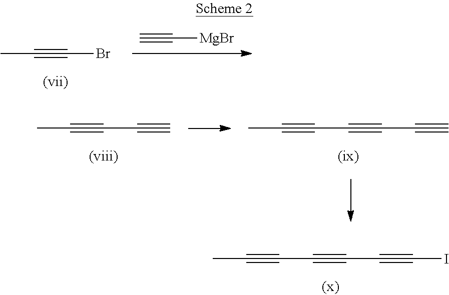

1-Bromoprop-1-yne (vii) is reacted with ethynylmagnesium bromide to afford penta-1,3-diyne (viii), which is further converted to hepta-1,3,5-triyne (ix). Compound (ix) can be readily transformed to 1-iodobepta-1,3,5-triyne (x) under a basic condition (e.g., n-BuLi), followed by addition of an iodo compound (e.g. I₂).

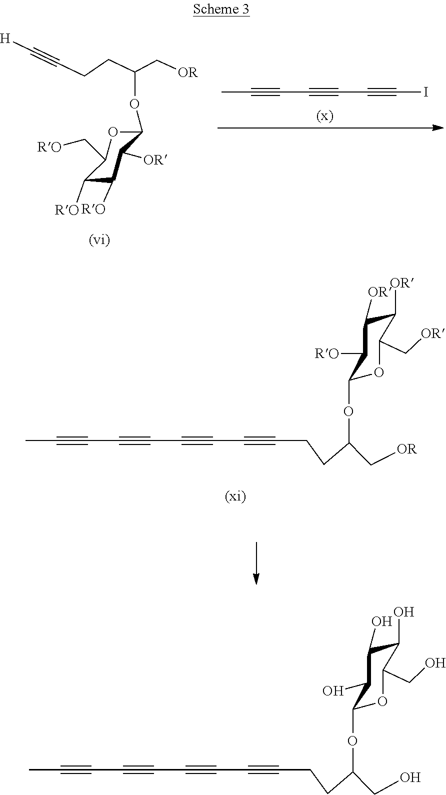

Scheme 3 demonstrates a coupling reaction between an acetylene derivative (vi), obtained from Scheme 1, and 1-iodohepta-1,3,5-triyne (x), obtained from Scheme 2, to a tetrayne compound (xi). Removal of protecting groups affords a polyacetylenic compound, 2β-D-glucopyranosyloxy-1-hydroxytrideca-5,7,9,11-tetrayne, a compound of this invention.

Synthetic chemistry transformations useful in synthesizing applicable compounds are described, for example, in R. Larock, Comprehensive Organic Transformations, VCH Publishers (1989); T. W. Greene and P. G. M. Wuts, Protective Groups in Organic Synthesis, 3^{rd} Ed., John Wiley and Sons (1999); L. Fieser and M. Fieser, Fieser and Fieser's Reagents for Organic Synthesis, John Wiley and Sons (1994); and Paquette, Encyclopedia of Reagents for Organic Synthesis, John Wiley and Sons (1995) and subsequent editions thereof.

This invention features a method of administrating an effective amount of one of the above-described polyacetylenic compounds or a Bidens pilosa preparation containing such a compound to a subject in need thereof.

A composition for oral administration can be any orally acceptable dosage form including capsules, tablets, emulsions, and aqueous suspensions, dispersions, and solutions. In the case of tablets, commonly used carriers include lactose and corn starch. Lubricating agents, such as magnesium stearate, are also typically added. For oral administration in a capsule form, useful diluents include lactose and dried corn starch. When aqueous suspensions or emulsions are administered orally, the active ingredient can be suspended or dissolved in an oily phase combined with emulsifying or suspending agents. If desired, certain sweetening, flavoring, or coloring agents can be added.

Bidens pilosa plants were collected from the campus of Academia Sinica, Taiwan. Approximately 10 kg of cleaned and crushed plants, in their entirety, was refluxed in 40 L of water for two hours. After removal of aqueous phase, insoluble materials was again refluxed in 25 L of water for two hours. The combined aqueous solutions (approximately 65 L) were evaporated in vacuo to yield a residue, which was subsequently suspended in 1.0 L of water and extracted with 1.0 L of n-butanol for three times. The n-butanol fraction was first evaporated on a vacuum rotary evaporator under reduced pressure and then lyophilized to provide a crude product of cytopiloyne (37.7 g).

The crude product was subsequently chromatographed over a RP-18 silica gel column with a CH₃OH/H₂ O gradient solvent system to give sub-fractions BPB1, BPB2, BPB3, and BPB4. The BPB3 fraction, eluted by 70% CH₃OH, was further fractioned by semi-preparative HPLC using a CH₃OH/H₂O solvent system. Cytopiloyne was obtained and characterized by ¹H NMR and ¹³C NMR.

¹H NMR (500 MHz, CDOD₃) δ 1.78 (2H, q, J=6.8 Hz), 1.98 (3H, s), 2.58 (2H, t, J=6.8 Hz), 3.19 dd, 9.1, 7.8 Hz), 3.30 (1H, m), 3.34 (1H, m), 3.59 (2H, m), 3.65 (1H, dd, J=12.0, 6.5 Hz), 3.75 (1H, p, 6.8 Hz), 3.85 (1H, dd, J=12.0, 1.7 Hz), 4.32 (1H, d, J=7.8 Hz); ¹³C NMR (125 MHz, CDOD₃) δ 3.8, 16.1, 31.4, 60.0, 60.9, 61.8, 62.4, 62.6, 64.9, 65.8, 66.2, 71.5, 75.2, 77.9, 81.6, 104.8.

Calculation of the percentage of Bidens pilasa powder (BPP) is as follows: Biden pilosa powder weight/Biden Pilosa powder weight+basic chicken feed=% of BPP.

By 0.0005%˜15% (w/w) it meant that all ten-thousandth, thousandth, hundredth, tenth and integer unit amounts within the range are specifically disclosed as part of the invention. Thus, 0.0001% 0.0002%, 0.0003% . . . 0.001%, 0.002%, 0.003% . . . 0.01%, 0.02%, 0.03% . . . 0.1%, 0.2%, 0.3%, 0.4%, 0.5%, 0.6%, 0.7%. 0.8%, 0.9% and 1%, 2%, 3%, 4% . . . 13%, 14%, and 15% unit amounts are included as embodiments of this invention.

The invention relates to the discovery of the effect of B. pilosa on growth performance, gut microbiota and gut pathology in the presence or absence of E. tenella infection in chickens.

In the interests of food safety and public health, plants and their compounds are now re-emerging as an alternative approach to treat gastrointestinal diseases in chickens. Here, we studied the impact of the edible medicinal plant, B. pilosa, on growth performance gut bacteria and coccidiosis in chickens. First, we found that B. pilosa significantly elevated body weight gain and lowered feed conversion ratio in chickens. Next, we showed that B. pilosa reduced cecal damage as evidenced by increased hemorrhage, villas destruction and villus-to-crypt ratio in chicken ceca. We also performed pyrosequencing of the PCR ampilcons based on the 16S rRNA genes of gut bacteria in chickens. Metagenomic analysis indicated that the chicken gut bacteria belonged to 6 phyla, 6 classes, 6 orders, 9 families, and 8 genera. More importantly, we found that B. pilosa affected the composition of bacteria. This change in bacteria composition was correlated with body weight gain, feed conversion ratio and gut pathology in chickens. Collectively, this work suggests that B. pilosa has beneficial effects on growth performance and protozoan infection in chickens probably via modulation of gut bacteria.

### EXAMPLES

Exemplary instruments, apparatus, methods and their related results according to the embodiments of the present invention are given below.

### Materials and Methods

### Preparation of Chicken Diets

Chicken diets were mixed with phosphate-buffered saline (PBS) vehicle or 0.5% B. pilosa (Chun-Yueh Biotech Company, Taiwan). Preparation of B. pilosa was processed as previously published (Yang et al. “Effect of Bidens pilosa on infection and drug resistance of Eimeria in chickens” Res Vet Sci 98: 74-81). Briefly, whole plant of B. pilosa was authenticated, processed and mixed with chicken feed.

### Animal Husbandry

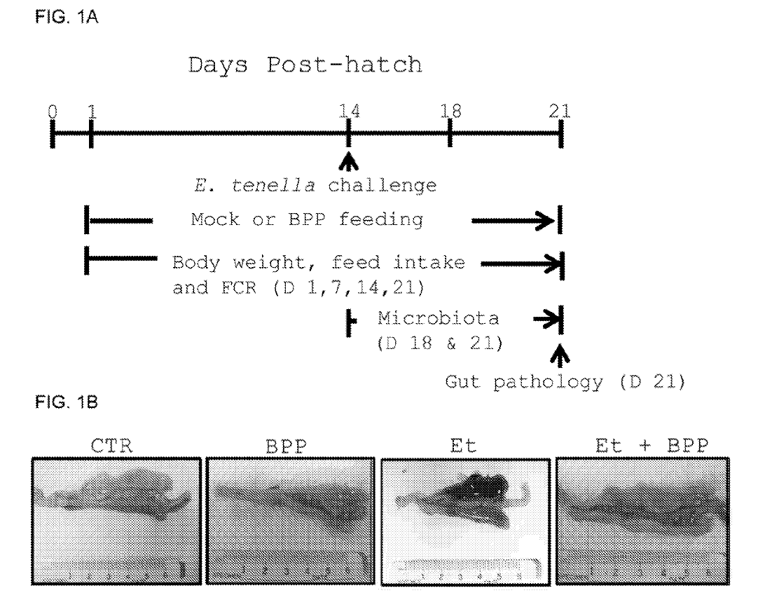

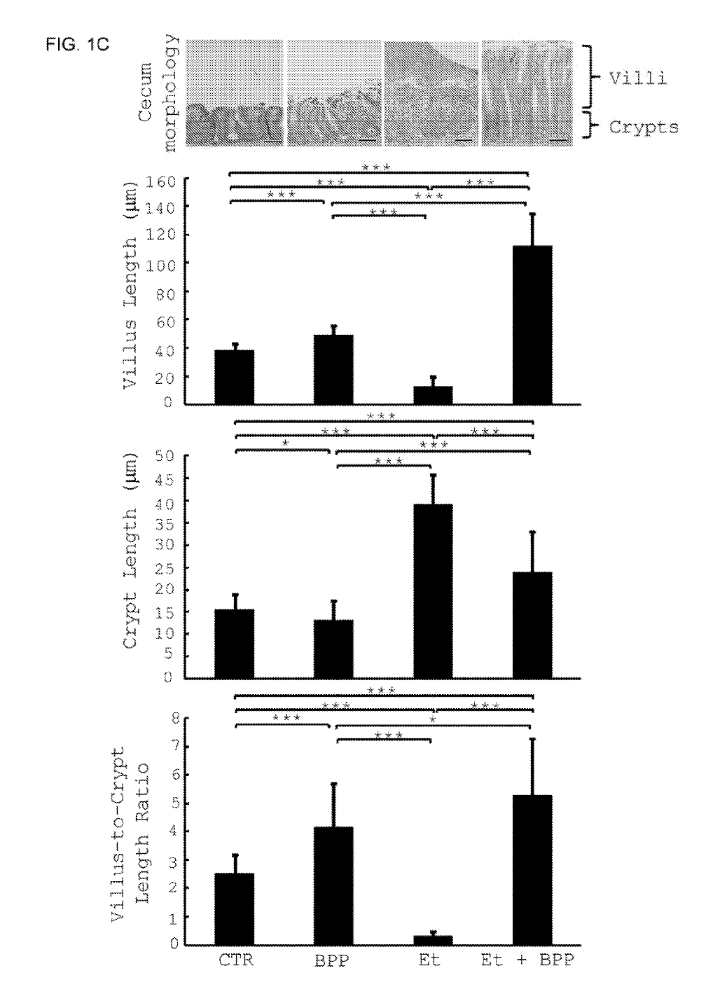

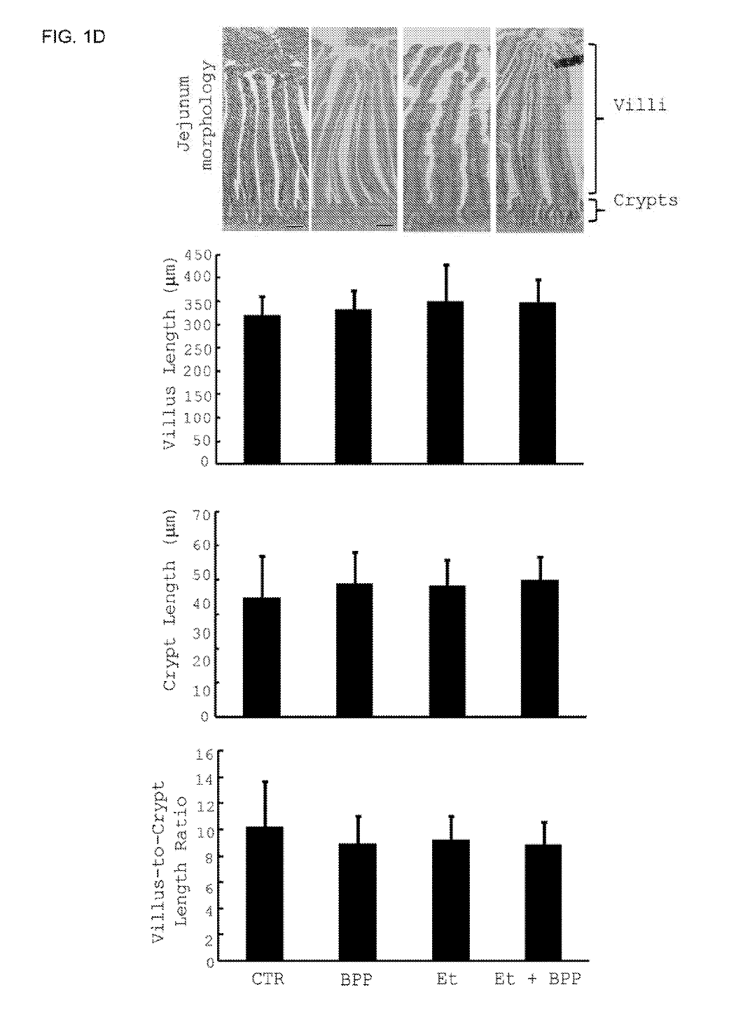

One-day-old disease-free Lohmann layer chicks were purchased from a local hatchery in Taichung, Taiwan. The birds were randomly divided into 4 groups. Each group was housed in three cages: Group 1 (3, 3, 4 chicks), Group 2 (3, 3, 4 chicks), Group 3 (3, 3, 4 chicks), and Group 4 (3, 3, 4 chicks). The chicks had free access to feed and water throughout the experiment. Group 1 (CTR) and Group 2 (Et) were fed with a standard diet whereas Group 3 (BPP) and Group 4 (Et+BPP) were fed with a standard diet containing 0.5% B. pilosa powder (5 g BPP/kg diet) from day 1 to day 21 (FIG. 1A). On day 14, Groups 2 and 4 were infected with E. tenella. The birds were raised in an institutional chicken house at 28-30° C. and handled according to the guidelines of the National Chung-Hsing University Institutional Animal Care and Use Committee. The protocol was approved by the same Committee. All surgery was performed under sodium pentobarbital anesthesia, and all efforts were made to minimize suffering.

### Preparation and Inoculation of E. tenella Oocysts

E. tenella strain Et C1 was maintained, amplified and used throughout the experiment as previously described. The oocysts were isolated from fresh feces of chickens, followed by sporulation with potassium dichromate. Four groups of birds, supplied with standard diets and standard diets containing 0.5% B. pilosa powder were tube-fed with 2 ml of sterile water (UI groups) or E. tenella sporulated oocysts×(1×10⁴, I groups).

### Measurement of Body Weight, Food Conversion and Gut Pathology in Animals

Each group of birds was individually weighed on a daily basis. Their diet consumption was monitored daily. Feed conversion ratio (FCR) was obtained by normalization of diet consumed by body weight. To evaluate gut pathology, the ceca and intestines removed from each group of sacrificed chickens were fixed with formalin and embedded with paraffin. The gut slides were stained with hematoxylin and eosin (HE) and examined under as microscope as described previously.

### Pyrosequencing and Data Analysis

The gut bacterial DNA collected from the feces of the chickens in Groups 1 to 4 on day 18 or 21 (i.e., day 4 and 7 post infection) were purified and used as templates for PCR amplification with 16S rRNA primers (F: 5′AGAGTTTGATCCTGGCTCAG3′ and R: 5′CGGTTACCTTGTTACGACTT3′). Following pyrosequencing (ROCHE 454™), chimeric sequences of the 16S rRNA sequences were removed using Chimera Check. The trimmed sequences over 300 bp were analyzed using RDPipeline as published. Briefly, 165 rRNA gene sequence alignment (Aligner), 16S rRNA gene sequence clustering (Complete Linkage Clustering), α-diversity index (Shannon index and Chaol estimator), rarefaction curve, and phylogenetic analysis (RDP classifier) were conducted. Principle component analysis and clustering analysis for bacterial genera were performed using the preomp, heatmap3 and ggplot2 functions in R (the R Foundation for Statistical Computing). The hierarchical multi-level pie charts of bacterial compositions of experimental samples based on phylogenetic classifications were constructed using KRONA software.

### Statistical Analysis

Data from 10 chickens in each group of chickens are presented as mean ±standard error (SE). ANOVA was performed to determine whether there was a significant difference between treatment groups and control groups. Actual P values are presented in all experiments. Spearman's rank correlation coefficient was used to test the association between microbiota and gut pathology and growth performance.

### Results

### B. pilosa Improves Growth Performance and Lowers FCR in Control Chickens and Those Infected with E. tenella

The benefits of this plant for growth performance in chickens were evaluated. We first monitored the body weight gain and FCR of chickens fed with a standard diet containing vehicle and 0.5% BPP (Table 1). We found that chickens fed with B. pilosa had a better body weight gain than those with a standard diet (Groups 1 and 3, Table 1). Consistently, B. pilosa significantly decreased FCR in chickens (Groups 1 and 3, Table 1). Next, we assessed the effect of B. pilosa on body weight gain and FCR in the chickens infected with E. tenella. We found that B. pilosa significantly augmented body weight gain and reduced FCR (Groups 2 and 4, Table 1). The data collectively demonstrated that B. pilosa promoted weight gain and diminished FCR in the presence or absence of E. tenella infection. Table 1 shows the effects of B. pilosa on body weight of chickens before and after E. tenella challenge.

TABLE 1

| | Bodyweight gain (%) | | | FCR | | |
|---|---|---|---|---|---|---|
| Group¹ | Day 14-21 | P value | P value | Day 14-21 | P value | P value |
| 1 (CTR) | 48.8 ± 3.2 | | | 3.14 ± 0.27 | | |
| 2 (Et) | 31.6 ± 11.5 | 0.0052 | | 5.16 ± 1.23 | 0.0012 | |
| 3 (BPP) | 52.0 ± 1.6 | 0.0354 | 0.0017 | 2.81 ± 0.08 | 0.0097 | 0.0003 |
| 4 (BPP + Et) | 45.5 ± 5.8 | >0.05 | 0.0200 | 3.71 ± 0.64 | >0.05 | 0.0176 |

¹The chickens were given standard diet (Groups 1 and 2) or standard diet supplemented with 50 g/kg diet Bidens pilose powder (Groups 3 and 4) from days 1 to 21. On day 14, chickens in Groups 2 and 4 were orally inoculated with E. tenella at a dose of 1 × 10⁴ sporulated oocysts per chicken. P values are indicated.

### Effect of B. pilosa on Gut Pathology Associated with E. tenella Infection

We checked the effect of gut pathology in 4 groups of chickens. Gross examination showed that the ceca of the chickens fed with a standard diet and a diet containing B. pilosa, without E. tenella infection, appeared to be similar (CTR and BPP, FIG. 1B). Microscopy showed that B. pilosa seemed to have longer villus length, shorter crypt length and, in turn, higher villus-to-crypt ratio in chicken coca (CTR and BPP, FIG. 1C). However, no difference in the structure of villi and crypts in the jejuna of chickens fed with or without B. pilosa was observed (CTR and BPP, FIG. 1D). Further, we examined the gut pathology in the chickens infected with E. tenella. We found that the ceca of the chickens infected with E. tenella were damaged with hemorrhaging and loss of cecal villi, 7 days post Eimeria infection (Et, FIG. 1B). Accordingly, microscopic examination indicated that E. tenella destroyed villi, increased crypt length and, in turn, reduced the villus-to-crypt ratio in the chicken ceca (Et, FIG. 1C), but not in chicken jejuna (Et, FIG. 1D). In contrast, B. pilosa reversed the damage caused by E. tenella and, therefore, increased villus length, decreased crypt length and augmented the villus-to-crypt ratio in chicken cera (Et+FIG. 1C). Overall the data showed that B. pilosa reduced E. tenella-dependent damage in chickens via gut modulation. Thus, B. pilosa alleviates E. tenella-mediated gut pathology in chickens.

### Overview of Chicken Gut Microbiota in 8 Experimental Settings

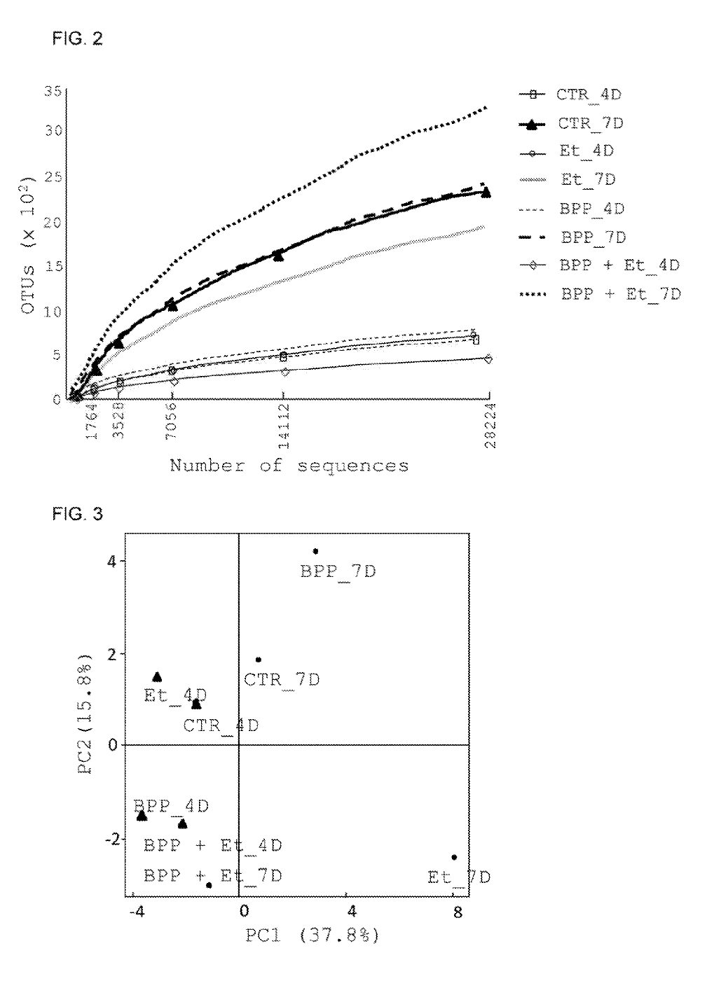

Next, we analyzed the effect of B. pilosa on gut bacteria in each group of chickens. Pyrosequencing-teased metagenomic analysis was conducted to uncover the bacterial communities in the guts of chickens aged 18 (D4) or 21 days (D7). A total of 200, 250 16S rRNA gene sequences were produced from 8 experimental samples. The number of sequences, operational taxonomic units (OTUs) and diversity indices for each sample are summarized in Table 2. Rarefaction curves suggested that the number of sequences from 8 experimental samples were enough to uncover major OTUs (FIG. 2). The gut microbiota of 21-day-old chickens (7D samples) are much more diverse than those of 18-day-old chickens (4D samples) as evidenced by Shannon and Chao1 diversity indices in Table 2 and curves in FIG. 2. Table 2 lists the number of sequences, OTUs, classification and diversity indexes for each sample.

TABLE 2*

| Sample DNA | Seq No. | OTUs | Phylum | Class | Order | Family | Genus | Shan | Chao1 |
|---|---|---|---|---|---|---|---|---|---|
| CTR_4D | 17048 | 595 | 4 | 9 | 9 | 14 | 23 | 4.27 | 823.4 |
| CTR_7D | 26031 | 2336 | 3 | 9 | 9 | 14 | 27 | 4.95 | 3837.2 |
| BPP_4D | 24305 | 682 | 4 | 8 | 9 | 13 | 21 | 4.20 | 927.8 |
| BPP_7D | 33008 | 2406 | 5 | 12 | 11 | 15 | 25 | 5.64 | 3563.7 |
| Et_4D | 22228 | 630 | 4 | 8 | 9 | 13 | 22 | 4.07 | 793.7 |
| Et_7D | 28806 | 1990 | 5 | 12 | 13 | 19 | 30 | 5.30 | 2874.7 |
| BPP + Et_4D | 20844 | 499 | 4 | 11 | 12 | 15 | 24 | 3.56 | 713.7 |
| BPP + Et_7D1 | 27980 | 3300 | 4 | 11 | 12 | 16 | 28 | 5.58 | 5077.8 |

*OUT, Operational taxonomic unit.; Shan, Shannon diversity index, Chao1, Chao1 diversity index

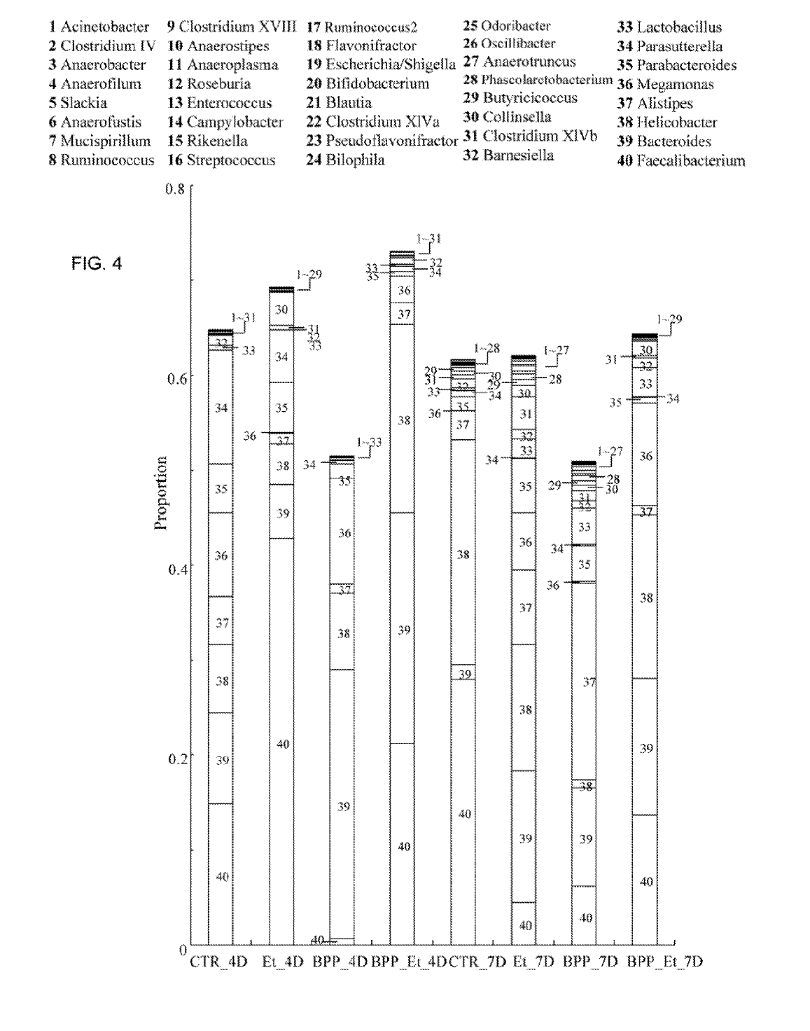

The sequence analysis using the RDP classifier revealed that 6 phyla, 13 classes, 15 orders, 25 families, and 42 genera of known bacteria were present in the samples. Overall, six phyla (Firmicutes, Bacteroidetes, Proteobacteria, Actinobacteria, Tenericutes and Deferribacteres), six classes (Clostridia, Bacteroidia, Epsilonproteobacteria, Negativicutes, Bacilli and Betaproteobacteria), six orders (Clostridiales, Bacteroidales, Campylobacterales, Selenomonadales, Lactobacillales and Burkholderiales), nine families (Ruminocoecaceau, Helicobacteraceae, Bacteroidaceae, Lachnospiraceae, Rikenellaceae, Veillonellaceae, Porphyromonadaceae, Lactobacillaceae and Sutterellaceae) and eight genera (Faecalibacterium, Helicobacter, Bacteroides, Alistipes, Megamonas, Parabacteroides, Lactobacillus and Parasutterella) existed in all eight samples in different proportions. The results from principal component analysis indicated that the bacterial community compositions of the eight samples were diverse (FIG. 3). The bacterial community composition at the genus level shown in FIG. 4 further confirmed the data obtained from the principal component analysis.

The chickens from Groups 1 to 4 were sacrificed on days 4 (D4) and 7 (D7) and the bacterial DNA samples of their guts (ceca and intestines) were pooled into 8 samples. Individual bacterial community compositions were analyzed. Details about the bacterial community compositions in eight samples were recorded (data not shown). To identify the co-occurring bacterial genera groups among eight experimental samples, clustering analysis was performed.

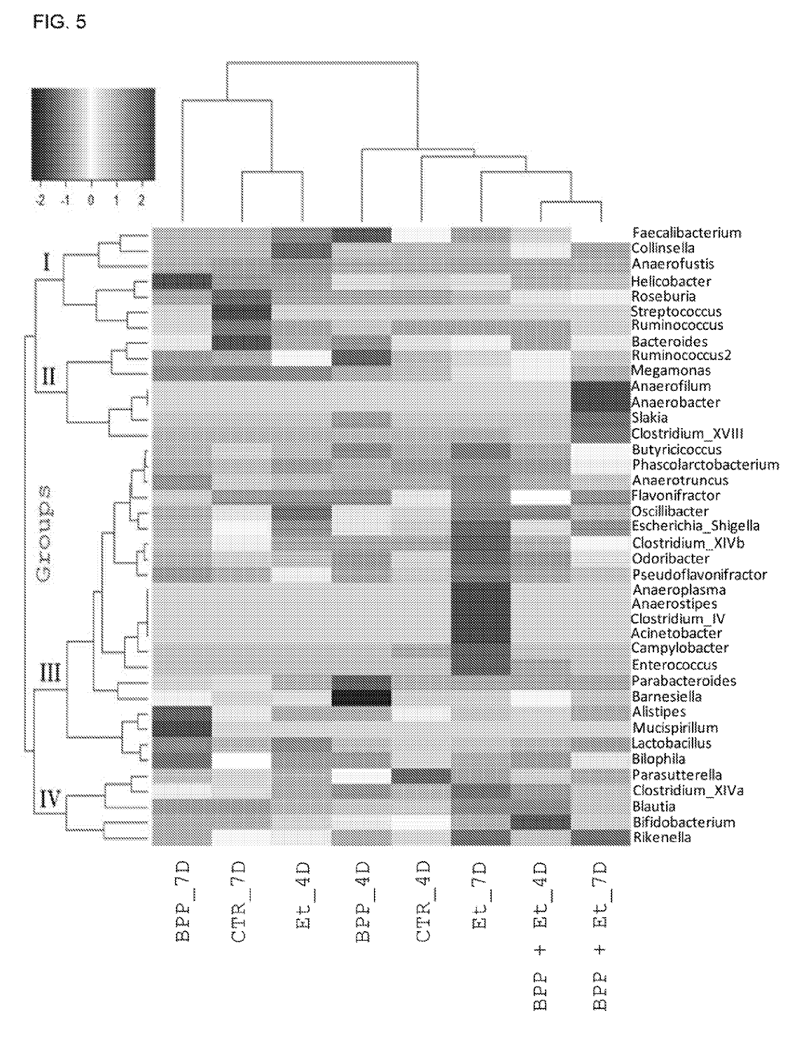

As shown in FIG. 5, 40 bacterial genera were grouped into four clusters: I to IV. Subsets of bacterial genera associated with growth performance and E. tenella infection in chickens were identified and described below.

### Effect of B. pilosa on the Change in Gut Microbiota

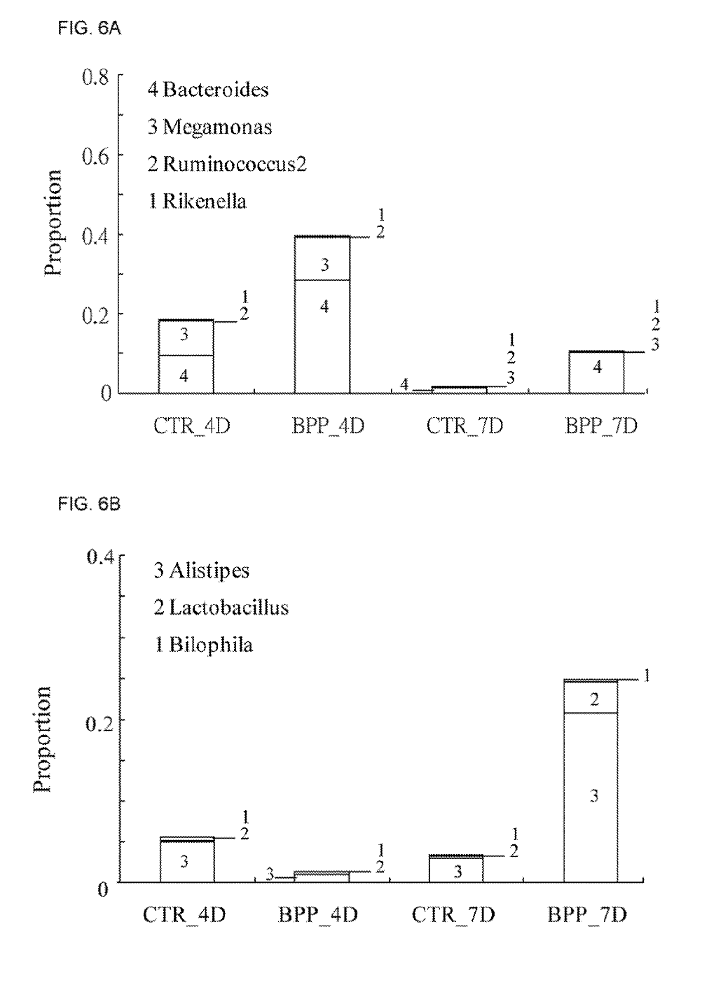

Gut microbiota have been documented to correlate to growth performance and gut health in chickens. We investigated the correlation between microbiota and growth performance in chickens fed with B. pilosa. Two subsets of bacterial genera in Group II and III were found to exhibit higher proportions in the guts of the chickens fed with standard diet containing B. pilosa but lower proportions in those of the other groups (FIG. 5). The first subset of bacterial genera, Bacteroides, Megamonas, Rikenella, and Ruminococcus2, was increased in B. pilosa-fed dickens aged 18 days (FIG. 6A). Similarly, the second subset of bacterial genera, Alistipes, Bilophila and Lactobacillus, was elevated in B. pilosa-fed chickens aged 21 days (FIG. 6B). All the above genera were reported to be beneficial microbiota. We failed to identify an elevation of these genera in the guts of chickens following E. tenella infection (data not shown) probably because the probiotics are easily disturbed by coccidiosis. Collectively, B. pilosa elevated a number of gut probiotics in chickens. Moreover, this elevation was inversely associated with FCR in chickens based on Spearman's rank correlation coefficient (r=−0.8 to −1).

### Gut Pathology-Associated Bacterial Genera after E. tenella Infection

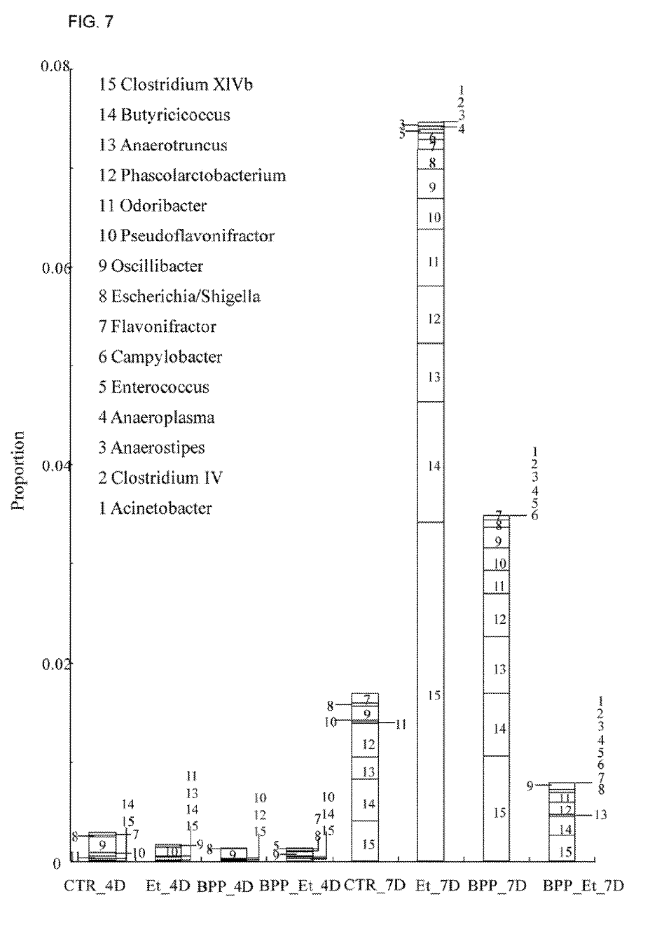

We also wanted to correlate microbiota with gut lesions in the guts of chickens, fed with PBS and B. pilosa, following E. tenella infection. Cluster analysis showed that one subset of 15 bacterial genera in Group III exhibited higher proportions in the guts of the E. tenella-infected chickens as they aged but lower proportions in the B. pilosa-fed E. tenella-infected chickens (Et_7D vs BPP+Et_7D, FIG. 5). Change in the proportion of the 15 bacterial genera in the guts of chickens, aged 18 days, 4 days post-infection was not evident (Et_4D vs BPP+Et_4D, FIG. 7). However, this change became evident in E. tenella-infected chickens, aged 21 days, suggesting that the 15 bacterial genera in chicken guts were associated with gut pathology (Et_7D vs BPP+Et_7D, FIG. 7). The 15 genera bacteria that decreased included Actinobacter, Clostridium IV, Anaerostipes, Anaeroplasma, Enterococcus, Campylobacteria, Flavonifractor, Escherichia/Shigella, Oscillibacter, PseodoFlavonifractor, Odoribacter, Phascolarctobaterium, Anaerotruncus, Butyricicoccus and Clostridium XIVb. Among them, Escherichia/Shigella, Campylobacter, Enterococcus, Clostridium and Acinetobacter are known as opportunistic pathogens of an zoonotic origin, that not only affect the domestic animal industry but also cause public health problems in humans. B. pilosa reduced the proportion of these opportunistic zoonotic pathogens in the guts of the chickens, suggesting this plant inhibited the pathogenic bacteria in the guts of chickens infected with E. tenella (FIG. 7). Moreover, the decrease in the above 5 harmful genera was inversely correlated with villus length and the villus-to-crypt ratio, hallmarks of gut pathology, in chickens based on Spearman's rank correlation coefficient (r=−0.8 to −1).

Here, we showed that B. pilosa enhanced growth performance (Table 1), changed gut microbiota (Table 2 and FIG. 5) and reduced E. tenella-implicated gut pathogenesis (FIGS. 1C and 1D). In addition, B. pilosa selectively increased probiotics and decreased harmful bacteria in the guts of chickens (FIGS. 5 to 7).

Our data collectively suggest that B. pilosa regulates a shift in gut microbiota in chickens. We found that B. pilosa altered the proportion of gut microbiota in chickens, including an increase in 7 probiotic genera (FIGS. 5 and 6) and a decrease in 15 bacterial genera, including 5 harmful bacteria (FIGS. 5 and 7). As far as the 7 probiotics are concerned, Alistipes, Bacteroides, Lactobacillus, and Ruminococcus are known as probiotics for growth performance and weight gain in chickens. Bacteroides and Megamonas were reported to be implicated in propionate production in chicken guts. Megamonas and Ruminococcus were reported to be involved in polysaccharide degradation and utilization in chicken guts. Bacteroides and Lactobacillus were shown to produce some essential vitamins (i.e., vitamin K, vitamin B12, and folic acid) and contribute to intestinal bile acid metabolism and recirculation. Moreover, Lactobacillus has been used as a probiotic to control coccidiosis in chickens infected with Eimeria species. Thus, gut microbiota play an important role in the clinical outcomes of coccidiosis in chickens. In sharp contrast, the proportion of 15 bacterial genera in chicken guts was decreased by B. pilosa (FIG. 7). Among them, Escherichia/Shigella, Campylobacter, Enterococcus, Clostridium and Acinetobacter, known as opportunistic pathogens of zoonotic origin, not only affect domestic animal industry but also cause public health problems in men. Consistent with the function of B. pilosa in outgrowth of probiotics, this plant prevented growth of these opportunistic zoonotic pathogens in chicken guts (FIG. 7). The data indicate that B. pilosa acts as a prebiotic to enhance the growth of probiotics in chicken guts to increase growth performance. B. pilosa may thus be useful as a feed substituent and additive, as our results show that it can improve growth performance (Table 1 and FIG. 1). This application also lowers feed cost from crops and anti-coccidial agents, and risk of anti-coccidial contamination. This work also expands the medicinal utility of B. pilosa in animals, to target the balance of gut microbiota.

In conclusion, we demonstrated the beneficial effect of B. pilosa on growth performance (i.e., body weight gain and FCR), gut bacteria and E. tenella infection in chickens. Overall the data suggest that B. pilosa may have a novel function as a prebiotic, which elevates beneficial bacteria and reduces harmful bacteria in chicken guts. This work further illustrates the potential use of B. pilosa as a feed additive in organic chicken production.

The foregoing description of the exemplary embodiments of the invention has been presented only for the purposes of illustration and description and is not intended to be exhaustive or to limit the invention to the precise forms disclosed. Many modifications and variations are possible in light of the above teaching. The embodiments and examples were chosen and described in order to explain the principles of the invention and their practical application so as to enable others skilled in the art to utilize the invention and various embodiments and with various modifications as are suited to the particular use contemplated. Alternative embodiments will become apparent to those skilled in the art to which the present invention pertains without departing from its spirit and scope. Accordingly. the scope of the present invention is defined by the appended claims rather than the foregoing description and the exemplary embodiments described therein.

All references cited and discussed in this specification are incorporated herein by reference in their entireties and to the same extent as if each reference was individually incorporated by reference.

1. A method for promoting beneficial gut microbiota and/or inhibiting pathogenic gut microbiota in an animal in need thereof, comprising:
administering to the animal in need thereof a composition comprising a therapeutically effective amount of Bidens pilosa extract, or an active compound isolated from the Bidens pilosa extract.

2. A method for promoting beneficial gut microbiota and/or inhibiting pathogenic gut microbiota in an animal in need thereof, wherein the animal is not afflicted with coccidiosis, comprising:
administering to the animal in need thereof, wherein the animal is not afflicted with coccidiosis, a composition comprising a therapeutically effective amount of Bidens pilosa extract, or an active compound isolated from the Bidens pilosa extract.

3. The method of claim 2, wherein the animal is in need of promoting gut health and/or growth performance.

4. The method of claim 2, wherein the animal is subjected to at least one of the following steps:
(i) performing examination of the animal gut health;
(ii) performing examination of the animal gut structure with X ray, CT scan, gut endoscopy;
(iii) performing examination of gut pathology of the animal; and
(iv) performing examination of crypt, villi, gut integrity, leukocyte infiltration, and/or inflammation.

5. The method of claim 2, wherein the animal is in need of gaining body weight.

6. The method of claim 2, wherein the animal is selected from the group consisting of humans, non-human mammals, fish, birds, and reptiles.

7. The method of claim 2, wherein the beneficial gut micribiota comprise at least one bacteria genus selected from the group consisting of Bacteroides, Megamonas, Rikenella, Ruminococcus2, Alistipes, Bilophila, and Lactobacillus.

8. The method of claim 2, wherein the pathogenic gut microbiota are at least one bacteria genus selected from the group consisting of Actinobacter, Clostridium IV, Anaerostipes, Anaeroplasma, Enterococcus, Campylobacteria, Flavonifractor, Escherichia/Shigella, Oscillibacter, PseodoFlavonifractor, Odoribacter, Phascolarctobacterium, Anaerotruncus, Butyricicoccus, and Clostridium XIVb.

9. The method of claim 2, wherein the composition is in a dosage form selected from the group consisting of oral, capsule, suppository, and parenteral.

10. The method of claim 2, wherein the active compound isolated from the Bidens pilosa extract is a polyacetylenic compound of formula (I): 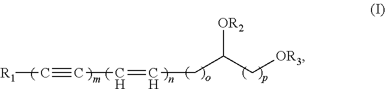
wherein
R₁ is H of CH3;
R₂ is monosaccharide;
R₃ is H or COCH₂COOH;
m=3 or 4;
n=0 or 1;
o=1 or 2; and
p=1 or 2.

11. The method of claim 10, wherein the active compound is selected from the group consisting of

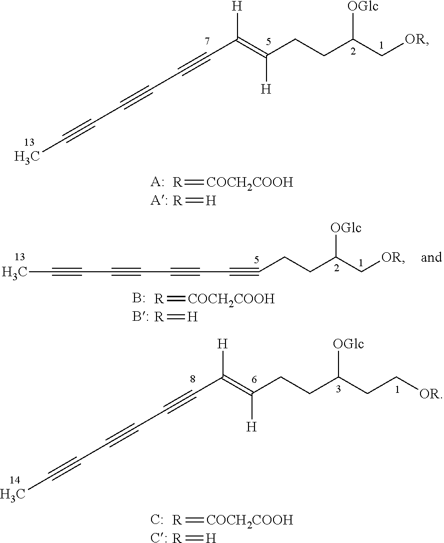

12. The method of claim 10, wherein the effective amount of the active compound isolated from the Bidens pilosa extract is at a dose of no less than 1 μg/kg body weight of the animal in need thereof.

13. The method of claim 2, wherein the composition comprises the animal feed and 0.0005%˜15% (w/w) of Bidens pilosa extract.

14. The method of claim 2, wherein the composition further comprises an animal feed.

15. The method of claim 2, wherein the Bidens pilosa extract is in a form of powder.

16. The method of claim 1, wherein the beneficial gut microbiota comprise at least one bacteria genus selected from the group consisting of Bacteroides, Megamonas, Rikenella, Ruminococcus2, Alistipes, Bilophila, and Lactobacillus.

17. The method of claim 1, wherein the pathogenic gut microbiota are at least one bacteria genus selected from the group consisting of Actinobacter, Clostridium IV, Anaerostipes, Anaeroplasma, Enterococcus, Campylobacteria, Flavonifractor, Escherichia/Shigella, Oscillibacter, PseodoFlavonifractor, Odoribacter, Phascolarctobacterium, Anaerotruncus, Butyricicoccus, and Clostridium XIVb.

18. The method of claim 1, wherein the active compound isolated from the Bidens pilosa extract is a polyacetylenic compound of formula (I): 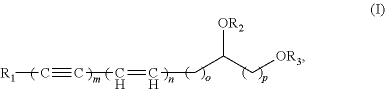
wherein
R₁ is H or CH3;
R₂ is monosaccharide;
R₃ is H or COCH₂COOH;
m=3 or 4;
n=0 or 1;
o=1 or 2; and
p=1 or 2.

19. The method of claim 18, wherein the active compound is selected from the group consisting of

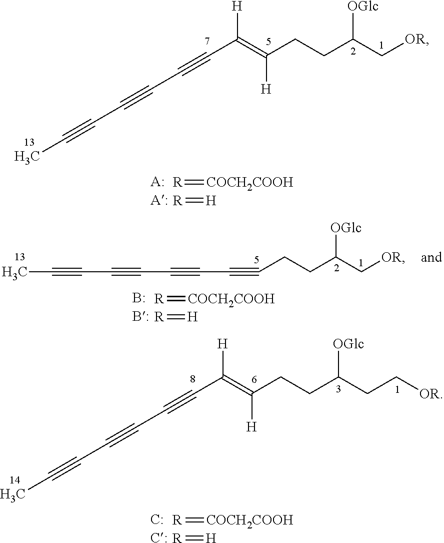

20. The method of claim 1, wherein the composition comprises the animal feed and 0.0005%˜15% (w/w) of Bidens pilosa extract.


SEQUENCE LISTING (2 sequences)

SEQ ID NO: 1 - DNA, 20 bp, Artificial Sequence
  16S rRNA primer Forward
agagtttgatcctggctcag

SEQ ID NO: 2 - DNA, 20 bp, Artificial Sequence
  16S rRNA primer Reverse
cggttaccttgttacgactt